(19) United States
(12) Patent Application Publication
(10) Pub. No.: US 2022/0339309 A1
(43) Pub. Date: Oct. 27, 2022

(54) Identification and elimination of micro-organisms in the air, on surfaces and on objects that are stationary or in motion using artificial intelligence and machine learning algorithms.

(71) Applicant: Keith Louis DeSanto, St Petersburg, FL (US)

(72) Inventor: Keith Louis DeSanto, St Petersburg, FL (US)

(21) Appl. No.: 17/241,540

(22) Filed: Apr. 27, 2021

(51) Int. Cl. A61L 2/24; G06N 20/00; G06N 5/04; A61L 2/22; A61L 9/14

(52) U.S. Cl. CPC A61L 2/24; G06N 20/00; G06N 5/04; A61L 2/22; A61L 9/14; A61L 2101/44

(57) ABSTRACT

Identification and elimination of micro-organisms in the air, on surfaces and on objects that are stationary or in motion using artificial intelligence and machine learning algorithms.

Identification and elimination of micro-organisms in the air, on surfaces and on objects that are stationary or in motion using artificial intelligence and machine learning algorithms.

This application focuses on elimination of the Micro-Organisms after specific identification has been made by the artificial intelligence platform.

### Accurate Elimination of Micro-Organisms—Machine Learning

Micro-Organisms are micro-scopic organisms that are too small to be visible to the human eye. The major groups of Micro-Organisms are bacteria, fungi, viruses, algae and protozoa. To see them, a high magnification lens (hi-def lens) is needed. Micro-Organisms have been studied for quite some time regarding their effects on people, plants and animals. They are airborne, are found on surfaces and on objects in motion. Micro-Organisms can be found on the moon (Streptococcus mitis), can survive (radiation) a nuclear explosion (Deinococcus radiodurans) and are on most surfaces including surfaces on objects that are in motion. Some are beneficial, some cause sickness and life-threatening diseases. Some of the beneficial Micro-Organisms help the human body such as probiotics. Probiotics are live bacteria that live in the stomach. Probiotics are found in fermented foods that, when consumed, take up residence in the stomach and improve health with digestion and also keep the bad bacteria in check.

The Micro-Organisms that are threats to our health or the lifestyle invaders are SARS, Ebola, E-coli, Borrelia burgdorferi (causes Lyme's Disease) and MRSA to name a few.

Identification of Micro-Organisms. Machine Learning, the past, the present and the future.

Our Artificial Intelligence “A1” platform with specific Machine Learning “ML” algorithms have learned about Micro-Organisms from the past (data gathering), the present (epidemics, pandemics and endemics) and are evaluating data and learning about their behaviors to predict what may be in store for the future. In order to accurately eliminate Micro-Organisms, identification must be fast, cheap to perform and accurate on all objects including those in motion. Learning about their behaviors may lead to an early warning about an upcoming threat.

The AI Platform defined.

The AI Platform is defined as an artificial intelligence platform utilizing hardware components, machine learning algorithms and artificial intelligence algorithms (software components) to understand, identify and eliminate Micro-Organisms. The hardware components can be nano-technology sized, normal sized (contemporary) or a combination of both. The hardware consists of a desktop computer, a server (or connected servers around the world), high magnification lenses (such as those in an electron microscope, a light microscope, an X-ray machine or NMR machine) drones and robots (some drones and robots equipped with Light Microscopes and Electron Microscopes) with and without mechanical arms, sensors, lasers, conveyor belts, bluetooth, satellite, flashing lights (Morse code), wireless and wired connections, voice to text, text to voice, picture to text or voice, voice or text to picture, all being powered by a single source or a combination of the following power sources: electricity, natural gas, hydrogen fuel cells, nuclear power, gasoline or diesel combustion engines, batteries, solar power, wind power, water power, where the AI platform is transportable, mobile or stationary. The algorithms can be singular or numerous all working together. They can be on separate desktops and servers, on numerous desktops or servers that are connected (in close vicinity or in other country) where the algorithms have 5 distinct purposes.

To compile data on Micro-Organisms including every facet of them (size, shape, color(s), possibly age and their ability to replicate quickly) Weight may also be needed at times.

To learn about their behaviors,

To understand why they behave in the wat they do,

To make decisions regarding indemnity, elimination and make warnings to synthetic germs and Micro-Organisms that may be unknown to man,

To predict and forecast events and behaviors of Micro-Organisms and germs (synthetic or not).

The complied data can be from the past (whether or not gathered from the AI Platform (hardware/software from other third-party databases) or present data (whether or not gathered from other forms of data bases).

Learning the behaviors of the Micro-Organisms by our algorithms leads to accurate identification as well as accurate elimination of them.

Drones, robots, lasers, conveyor belts, CPU's, glass slide positioning and electrostatic charge, sensors and hi def lenses are all managed by the AI Platform to identify Micro-Organisms accurately. This application makes the process faster, easier, cheaper and accurately eliminates microbe threats to humans, plants and animals. Contemporary (of this time period) identification applications utilize labs and stationary hardware that are time consuming, (transporting the Micro-Organism to a lab) costly (travel costs as well as permits to transport Biosafety Level 2, 3 and 4 “BSL level II, III and IV” “hazardous Micro-Organisms”) then to wait again for results such as those from a petri dish (Agar plates).

The new application works in the following manner. After identification of micro-organism, the artificial intelligence platform through its machine learning algorithms determines the type of micro-organism and the level of threat to humans, plants and animals. Threat level 10 is the identification of biological germs (Anthrax/Ricin) and viruses (SARS) that cause disease and death while threat level 1 is the identification of allergens such as cockroach excrement. Identification could be in the form of single or clusters of Micro-Organisms. Singular identification from the algorithms show data in rods, spheres, clusters, shapes, color, size, weight, circumference, diameter length width and height in 2 (x and y axis) and 3 (x, y and z) dimensions.

Specific Micro-Organism Identification is determined by size, shape and certain color(s). An example is a spore of pollen (about a 100 microns) to the Influenza virus (about 100 nano-microns). Another example is the particular color of a virus, while the Covid-19) virus has a red crown, the influenza virus has a blue and red crown. Data from the lenses is downloaded in several ways:

through drones and robots imbedded with hi-def lenses and their condensers with drones and robots having wireless connections,

downloads can take place wirelessly or wired from stationary AI Platforms,

manually by hand via storage devices-backup disks (over 1 terabytes), SD cards, USB drives or directly from the drone or robot that is directly downloaded by docking to the AI platform. Stationary lenses can also transmit the data via wired or wirelessly directly to the AI platform. If a picture of a cluster is captured and the file is large (over 100 megabytes in size), the image can be transmitted in pieces and assembled at the other end by the AI platform.

### Steps Before Elimination, Data Capture

Before elimination of Micro-Organisms can take place, there are many data points that need to be obtained, complied, calculated, compared to prior data, striped of certain characteristics and possibly forecasted. Micro-Organisms are detected while they are in motion or at rest.

### Data Required for Accurate Elimination

Behavior data such as “foot traffic” (amount of people that live/work/visit the residence/business/facility during a time period) is an example of data that needs to uploaded to the AI platform. People and animals transport Micro-Organisms to and from residences and businesses. Other delivery types are also required such as overnight courier deliveries, deliveries to warehouses, food and merchandise, and other basic delivery of materials. These specific types of data can be uploaded into the AI platform manually. Some versions of the AI platform utilize the video surveillance of the premises to compile such data.

### Uploading of Data

The data obtained is from:

Hardware,

Software

Human voice, text and description of pictures.

The following ways of uploading the data to the AI platform are:

Drones, robots, hi def lenses (light microscopes, electron microscopes and when required X-ray machines), environmental sensors and lasers use wired, wireless, voice and text to upload the data to the AI platform.

Data can be manually uploaded:

by hand using a backup disk (western digital) USB drive or SD card (The SD card can be manually pulled from the drone, robot or hi def lens storage mechanism and placed into the AI platform).

By text or voice, or

A combination of all three.

Some data can be uploaded manually but, most of the data is obtained by the AI platform.

The most basic data is what is the foot traffic in and out of the structure. This type of data can be uploaded through a connection to the residences or the businesses security system that has cameras or manually. Other data may be uploaded through text or voice. Some of the other data can also be uploaded by voice such as temperature and humidity, but also can be uploaded via text. The AI Platform uses all.

The data obtained from the hardware components must be transferred into the AI platform. The data is usually transferred wirelessly from the drones, the robots, the sensors, the lasers, and the lenses to the AI platform where the data is analyzed. Drone and robot docks on the platform can be used or the downloading of data can be made through wireless connection to the AI platform.

At times the data is downloaded directly from the drones, robots, (equipped with sensors and lasers) by physically docking to the AI platform.

Data points that should be obtained by the AI platform are:

Are animals present, what type of animal and how many.

How many floor or ceiling fans are in structure and how many are in each room

Where is the area geographically and what is the current weather. The AI platform uses GPS

Is the climate humid or dry.

What is located inside the structure/surrounding area. Example would be a chemical manufacturing facility. Are the chemicals toxic or natural such as a chlorine as opposed to a bamboo processing facility. This data is available from the internet where the AI platform has learned which buildings are businesses ad which are residences, what type of business is it.

Is there direct sunlight

How many windows and are there blinds or curtains

How many vents and where are they in the structure

Where are the heating and air-conditioning vents and how many

Temperature

Humidity

Surrounding effects (direct sunlight, artificial light, vents and what particular industry is housed in the structure and what industries are hereby).

Other data may be needed such as are there people who smoke? Any and all decisions are dependent on what the AI Platform initially and continually finds. After initial data gathering has begun, each instant at the onset of the engagement of the AI platform, there are calculations by the algorithms that either requests more information or has accurately detected a trend, matter or behavior of a cluster or single Micro-Organism.

The AI Platform requires data from the air, on surfaces, on stationary objects and on objects in motion. Different types of drones and robots and stationary platforms are utilized. Drones capture airborne Micro-Organisms using glass slides having an electrostatic charge.

Robots capture surface Micro-Organisms also using glass slides having an electrostatic charge. Data such as environmental conditions can be obtained from stationary platforms as well as different drones and robots equipped with sensors, lasers and hi magnification (hi-def) lenses. Micro-Organisms can also be obtained from moving objects such as a moving toy, a rotating fan blade or a tick or flea. The manner in which Micro-Organisms are captured from objects in motion is by simply touching the surface area of the moving object with a swab. The swab is held by a mechanical arm on a drone or robot. An instantaneous scrape or touch will do the job. Sometimes, an electrostatically charged plastic stick, plastic saran wrap on a swab can be used. Swabs and sticks can be miniature or larger than contemporary swabs being used today. The swab is then used to steak a glass slide for viewing with high magnification lenses such as an electron microscope, light microscope, X-ray machine or NMR machine (hi-def lenses). At times, we use a basic form of hi def lenses (stripped down version of a microscope eliminating the base and stage) by using just the lenses with the condenser, eliminating or replacing the stage and microscope base with a conveyor belt.

### Elimination of Micro-Organisms

Once the Micro-Organism has been identified, the AI Platform switches from identification to elimination mode. This is done by commanding all drones, robots to return to the AI platform and shut down. All sensors will also be closed down whether mobile or stationary. Different drones and robots will be used in the elimination process as opposed to identification drones, robots, lenses, sensors and lasers.

At this point, the AI Platform has already determined if the Micro-Organisms are singular or clusters thereof, what threat level they are and what is needed to eliminate them.

After the type and level of micro-organism is determined, the AI platform through machine learning algorithms have already determined what is needed through of database of billions of data points, complied data, behaviors, and predictions. The algorithms will direct the AI platform what application is needed (if any), how the application will be administered, what are the application mixtures, dilutions and what carriers are needed.

Different Micro-Organisms and pollutants, different elimination applications and behavior modifications.

Since there are many different pollutants, many different disease causing and life-threatening Micro-Organisms, there are many applications that can be applied with possibly many behaviors that need to be changed.

If the AI platform has identified a pollutant, many times an application will not be appropriate but a change in behavior is required. Also, the elimination of something needs to take place without any application.

### Elimination Applications and Behavior Modifications are Listed Below

The AI platform has come to many conclusions that some applications produce more problems with initial elimination undertakings and some behavior modifications cause more unhealthy conditions in residences and places of business. The machine learning algorithms constantly learns not only about Micro-Organisms and their behaviors but, their surroundings and the effects certain applications have had and what are the collateral results of such.

List of Pollutants that our AI platform can identify and learn from:

Asbestos

Formaldehyde

Radon Gas

Tobacco Smoke

Bacteria

Mildew, Mold,

Allergens, pollen, dust mites, cockroach, rat mice excrement.

Construction and hobby materials-paints, varnishes, glues, and cleaning products

Our AI platform utilizes elimination applications ranging from liquid chemicals, to gas and solids with numerous ratios of dilutions, to carriers and specific materials all of which are determined by the AI platform. Most of the elimination applications for pollutants are not needed where elimination of the polluting product is required or a change in behavior is mandated by the AI platform. An example is when a cleaning product is used in bathrooms such as bleach or chlorine is used to treat an indoor pool, a simple ventilation system may be needed to correct the problem.

### Micro-Organism Elimination

There are three ways that our AI platform elimination application works: by electric zapping or laser zapping of each Micro-Organism directed by the AI Platform. AI directs the laser to obliterate a single Micro-Organism, or a cluster. The AI platform may also direct the heating of flowing air to 200 degrees Fahrenheit and direct it close to surfaces to kill all Micro-Organisms without using any other application. This application can be used in an industrial complex where machinery is made of metal and can withstand high temperatures and the concrete ground can also withstand the heat without degradation.

The second way is to break apart the cell wall of the Micro-Organism using chemicals administered by drones and robots.

### Surfactants

Some surfactants work well with eliminating Micro-Organisms. Surfactants are synthetic (man-made) using petroleum (oil). Biosurfactants, are made from nature and can be learned about from the inventors prior USPTO biofilm and rhamnolipid applications.

### Biosurfactants

Rhamnolipid Biosurfactants are secreted from the bacterium Pseudomonas aeruginosa. Since Pseudomonas aeruginosa is found everywhere (on most surfaces, in water on plants, animals and humans) they are ubiquitous. The correct way of stating rhamnolipid is without the “s” but, for this application the spelling of rhamnolipids will be used.

Rhamnolipid is quite easy to produce in small quantities as referenced in many colleges and universities throughout the world. The success to large scale rhamnolipid production has been the inability to reduce foaming, use a large container for production and maintain a continual exact result in each final production run. Our Miro-Organism rhamnolipid application has succeeded in eliminating those obstacles to large rhamnolipid production.

### Rhamnolipid Production

The rhamnolipid production procedure for our virus application uses the following steps:

We use a blend of three different Pseudomonas aeruginosa strains.

The components of our rhamnolipid production is listed below:

The production application described below uses closed plastic containers ranging from 5 gallons to 5,000 gallons and can be scaled up to over 100,000 gallons by using larger plastic tanks paddles, internal heaters, and oxygen tanks. The application uses a cement mixer paddle (covered in plastic) attached to a drill with the amount of revolutions of 100 per minute up to 800 per minute. The paddle used is a 24-inch quick mixer for the 100-revolution application The heater element is set to a constant 105 degrees Fahrenheit. We use pure oxygen in our production container.

The final product works well when mixed with peptides to eliminate several Micro-Organisms by breaking apart the cell wall of the Micro-Organism.

Applying rhamnolipid with specific ratios of mono-rhamnolipid to di-rhamnolipid is also determined by the AI platform.

### Virus Walls and the Piercing of

A virus is made up of a DNA or RNA genome inside a protein shell. Some viruses have an external membrane envelope such as the Corona Virus.

The envelope is made of lipids and proteins in the way a regular cell membrane is structured.

The interaction between antimicrobials is synergistic when the combined activity is greater than the additive effect of the antimicrobials. This chemical formula was specifically designed to be broad spectrum and highly effective against gram positive and gram-negative pathogenic bacteria, pathogenic fungus, pathogenic viruses (including COVID-19 and flu viruses) and degrade biofilms.

### Biosurfactant

Starting with the biosurfactant Rhamnolipid (RL). This allows the antimicrobial solution to better penetrate the virus cells using Rhamnolipid as its own delivery system. The biosurfactant acts as a detergent interacting with the virus membranes. Biosurfactant interacts with lipid bilayer of gram-negative bacteria increasing the negative charges on the cell surface which allows the cationic (positive charged) antimicrobial organic peptides to greater adherence and faster penetration into the microbes causing cytoplasm breakdown and quick cell death. Biosurfactants have the ability to remove the lipopolysaccharide membrane (LPS), disintegrate the cell membrane creating an opportunity for other ingredients to additively break down and permeabilize the cell wall and penetrate into the cytoplasm for an irreversible death.

### Antimicrobial Small Peptides

There are multiple mechanisms of action for these cationic (positive charge) groups. They are able to disrupt the membrane and when combined with other antimicrobials can penetrate the virus causing cytoplasmic disruption. The effect of these peptides are dependent on the ability of multiple charges to attach to and interact with the membrane of the virus. These charges are synergistically enhanced by biosurfactants and other peptides. There is further cell penetration by the organic acids in solution that attack the cell wall, chelate minerals and dissociate within the cell cytoplasm.

### Organic Acids

There are many antimicrobial organic acids. Some are considered weak. Weak acids are most effective in their undissociated form. This is because once inside the cell, the acid dissociates (goes into solution) because the cell cytoplasm (interior) has a near neutral pH. Protons generated from intracellular dissociation of the organic acid (H+) turn the cytoplasm acidic and must be removed by the organism. The cytoplasmic membrane is impermeable to H+ protons and must be actively transported to the exterior of the cell. This causes the cell to use tremendous energy to pump out the constant influx of these H+ protons which will eventually exhaust the micro-organism leading to death.

This solution combines organic acids allowing the pH to be more effective as the acids have different pKa values (where the acid is 50% in solution and 50% not in solution) and since each of the acids act upon gram-negative and gram-positive bacteria differently their combination allows for better cell membrane penetration. The complex combinations of organic acids create a synergistic reaction present as a powerful antimicrobial at low concentrations. Broad spectrum activity and is effective against bacteria, (both gram positive and gram negative) and viruses.

Our research shows that high purity rhamnolipids over 66% is for antimicrobials and lower purity under 65% has more surfactantcy. We use a combination of both for our anti-bacterial and anti-virus application.

Using peptides combined with rhamnolipids increases the success of the application.

Our initial research shows that rhamnolipid when induced into the human body can also stop the replication of Covid19 and eliminate the virus from replicating.

### Elimination of Molds and Mold Spores and the Like

Through our AI platform, identification of molds, mold spores, clusters of molds and clusters of mold spores with other Micro-Organisms are eliminated through the following applications:

Using drones and robots to spray a mist from their holding tanks to either break apart specific spores “lyse” or bring the spores to the ground to be clean up a clean robot or drone.

Peptides mixed at certain levels with biosurfactants with specific carriers lyse mold spores. Since there are over 100,000 different species of molds and many different types of peptides, there are many different specific elimination applications. When a molecule consists of between 2-50 amino acids, it is called a peptide. Lager chains of greater than 50 amino acids are referred to as proteins. When different peptides are mixed with different ratios of mono-rhamnolipids to di-rhamnolipids, this creates a specific elimination application to eliminate a specific micro-organism. Our machine learning algorithms have learned many different applications of peptides their carriers, their dilutions and biosurfactant combinations to eliminate specific Micro-Organisms. The AI platform with the machine learning algorithms determines the application after identification of the Micro-Organism, Micro-Organisms or clusters thereof.

Using the drones and robots to create air flow to heat the circulating air to a minimum of 120 degrees Fahrenheit to kill the spores in the air is also used as an application as determined by the AI platform. The tubes are heated and are at least 6 inches long so that enough heat is placed continually for least 1 second on a single or cluster of Micro-Organisms. On surfaces, the application can only be used where objects and furniture are not destroyed by the heat.

1. An application that identifies then eliminates viruses, bacteria, fungus and biological germs, in the air, on surfaces and on objects that are stationary or in motion using drones, robots, holding tanks for liquids, gases and powders, high magnification lenses and their condensers, sensors, lasers, machine learning algorithms and artificial intelligence.

2. An application that identifies then eliminates viruses, bacteria, fungus and biological germs, in the air, on surfaces and on objects that are stationary or in motion using drones, robots, holding tanks for liquids, gases and powders, high magnification lenses and their condensers, sensors, lasers, machine learning algorithms and artificial intelligence to determine which specific application is required for elimination, which combination of applications are required for elimination, which carriers are required for elimination and what dilutions are required for elimination.

3. An application in claims 1 and 2 where an artificial intelligence platform manages the entire elimination application through a list of steps and processes of which will be chosen, which specific ratios of mono-rhamnolipid to di-rhamnolipid are needed, if other biosurfactants are needed, if peptides are needed, which combination of carriers may be needed and if the elimination requires a single or combination of specific ingredients.

4. An application in claims 1 through 3 where the platform is completely autonomous.

5. An application in claims 1 through 3 where the sizes of the components are either nano-technology sized, normal “contemporary” sized or a combination of both.

6. An application in claims 1 through 3 where the application seeks out viruses, bacteria fungus and biological germs in places where the human eye can't see.

7. An application in claims 1 through 3 where a source of pollution is identified, the type of pollution is identified and the application and or process to eliminate the pollution is stated.

8. An application in claims 1 through 3 where the source of allergins is identified, the type of allergen is identified and the application and or process to eliminate the allergin is stated.

9. An application in claims 1 through 3 where the application eliminates enveloped viruses.

10. An application in claims 1 through 3 where the influenza virus is eliminated.

11. An application in claims 1 through 3 where a combination of many biosurfactants are used.

12. An application in claims 4 through 11 where an electrostatic spray is used to apply the application.

13. An application in claims 4 through 12 where the drones and robots utilize a tank attached to a misting machine that is used to apply the application.

14. An application in claims 1 through 13 where the result of the application is a biofilm that was left over from the application after it was administered.

15. An application in claims 1 through 14 where the application is managed by an artificial intelligence platform which include machine learning algorithms to determine the surrounding characteristics of the area utilizing past data, present data and predicting future data.

16. An application in claims 1 through 15 where the machine learning algorithms through the input of immediate data to determine which application is needed to eliminate viruses, bacteria, fungus, biological germs, pollution and or allergens
